(19) United States
(12) Patent Application Publication
(10) Pub. No.: US 2026/0305984 A1
(43) Pub. Date: Oct. 8, 2026

(54) SOFT WEARABLE PATCH FOR CONTINUOUS CARDIAC BIOMETRIC SECURITY

(71) Applicant: Georgia Tech Research Corporation, Atlanta, GA (US)

(72) Inventors: Woon-Hong Yeo, Atlanta, GA (US); Sung Hoon Lee, Atlanta, GA (US)

(21) Appl. No.: 19/481,810

(22) PCT Filed: May 15, 2024
(86) PCT No.: PCT/US2024/029401

§ 371 Date: Nov. 5, 2025

Related U.S. Application Data
(60) Provisional application No. 63/502,254, filed on May 15, 2023.

(51) Int. Cl. A61B 5/117; A61B 5/00; A61B 7/04

(52) U.S. Cl. CPC A61B 5/117; A61B 5/6833; A61B 5/7207; A61B 5/7246; A61B 5/725; A61B 5/7267; A61B 7/04; A61B 2562/166

(57) ABSTRACT

An exemplary embodiment of the present disclosure provides a biometric identification system, comprising one or more sensors and a controller. The one or more sensors can be configured to generate data corresponding to sounds generated by the heartbeat of a user. The controller can be configured to receive the data and authenticate the user based, at least in part, on the data.

### CROSS-REFERENCE TO RELATED APPLICATIONS

This application claims the benefit of U.S. Provisional Application Ser. No. 63/502,254, filed on 15 May 2023, which is incorporated herein by reference in its entirety as if fully set forth below.

### FIELD OF THE DISCLOSURE

The various embodiments of the present disclosure relate generally to biometric identification systems and methods.

### BACKGROUND

A biometric identification and security system can be described as a set of coordinated technologies that collect information about an individual and use that data to identify a person and unlock a pathway. It can be one of the most secure of the three categories of security, and the previously mentioned identification is made through any biological characteristic of an individual. Current measures of protection fall into one of three categories: a password system, scannable IDs, or individual biometric system. This can include but is not limited to fingerprints, facial structure, retina traits, iris attributes, and many more. In the broad umbrella of everything, there is an increasing necessity for better security and identification technology as advancements in science and engineering continue to be made. Security, like everything else, must keep up with the technology of the time. Yet, current biometrics have limitations, and their disadvantages create a technological gap, ranging up to $1,000 for more foolproof authentication devices. Present fingerprints, hand geometry, and voice recognition biometrics can be replicated and impacted by changing environmental factors. The required threshold for the correlation between incoming and known data must compensate for possible environmental factors. Currently, there is a magnitude of user resistance to Facial ID because, despite its advantages, there is some hesitancy because of privacy concerns. Because it scans for particular patterns, inconvenient lighting scenarios, and random reflections, iris scanning can be fooled with a high-quality image of a known user's eye. Because this biometric is more expensive than the rest, all these disadvantages add up to it being overpriced for inferior technology. One of the most crucial disadvantages for all security above applications is that there is no continuous monitoring across multiple security dimensions.

Behavioral biometrics have been examined for continuous authentication through smartphones, wearable devices, and facility cameras. Examples of such behavioral biometrics include keystroke and touchscreen dynamics, eye movement, walking gait, body gestures, and sounds of behaviors. However, these listed biometrics are subject to change over time, such as how an individual's eye movement dynamics and walking gait vary with time of day, stress, and other environmental factors. Studies of a user's behavioral evolution have been proposed but proved difficult due to the lack of training data sets and successful AI models. Another example of behavioral biometrics is keystroke dynamics, where machine learning models extract features from a user's raw keystroke data to allow or prevent authentication. Although this method improves the strength of typical passwords by discerning a user's unique typing tendencies, like other behavioral biometrics, keystroke dynamics have lower accuracy and shorter permanence than physiological characteristics such as heart sounds.

The advantages of biometrics are numerous and significant. The first of the advantages is that biometric data is difficult to forge, providing excellent security because only known users will be granted entry when using these systems. In addition, they do not require user memory or an actual key-biometric technology makes users themselves the key. With this type of security, there should never be a situation in which a user cannot get past the verification step. Another advantage is the automatic and easy identification of biometric systems. These key advantages are the most important for this technological era, and biometrics are currently considered bleeding-edge technology. It can already be seen in office buildings and hospitals.

Accordingly, there is a need for improved biometric identification/authentication systems and methods that address one or more of the disadvantages discussed above. Embodiments of the present disclosure can provide such systems and methods.

### BRIEF SUMMARY

An exemplary embodiment of the present disclosure provides a biometric identification system, comprising one or more sensors and a controller. The one or more sensors can be configured to generate data corresponding to sounds generated by the heartbeat of a user. The controller can be configured to receive the data and authenticate the user based, at least in part, on the data.

In any of the embodiments disclosed herein, the one or more sensors can comprise one or more MEMS microphones.

In any of the embodiments disclosed herein, the one or more sensors can be disposed on a flexible patch, and the flexible patch can be configured to be worn by the user.

In any of the embodiments disclosed herein, the controller can be configured to authenticate the user based, at least in part, on the data by: identifying one or more features in the data received from the one or more sensors; and comparing the one or more features to corresponding known features in sounds produced by the heartbeat of the user.

In any of the embodiments disclosed herein, the one or more features can correspond to S1 and S2 heart sounds of the user.

In any of the embodiments disclosed herein, the one or more features can correspond to one or more of magnitude, shape, size relativity between S1 and S2 sounds, and standard deviations in a timing duration of a cardiac cycle of the user.

In any of the embodiments disclosed herein, the controller can be further configured to bandpass filter the data.

In any of the embodiments disclosed herein, the bandpass filtering can occur from 20 Hz to 250 Hz.

In any of the embodiments disclosed herein, the controller can be further configured to zero-phase filter the data.

In any of the embodiments disclosed herein, the biometric identification system can further comprise an analog to digital signal converter configured to receive analog data from the one or more sensors and convert the analog data to digital data to be processed by the controller.

In any of the embodiments disclosed herein, the controller can be configured to employ a convolutional neural network (CNN) to authenticate the user.

In any of the embodiments disclosed herein, the CNN can be trained with data collected from the user.

In any of the embodiments disclosed herein, the controller can comprise one or more processors that work individually or collectively to perform the various functions of the controller.

In any of the embodiments disclosed herein, the controller can comprise a smartphone, and the smartphone can be configured to receive data from the one or more sensors wirelessly.

In any of the embodiments disclosed herein, the controller can further comprise a microcontroller mechanically coupled to the one or more sensors and configured to receive data from the one or more sensors and transmit data to the smartphone.

Another embodiment of the present disclosure provides a method of authenticating a user. The method can comprise: receiving data indicative of sounds generated by the heartbeat of a user; and authenticating the user based, at least in part, on the data.

In any of the embodiments disclosed herein, the data can be generated by one or more sensors.

In any of the embodiments disclosed herein, the one or more sensors can comprise one or more MEMS microphones.

In any of the embodiments disclosed herein, the one or more sensors can be disposed on a flexible patch, and the flexible patch can be worn by the user.

In any of the embodiments disclosed herein, authenticating the user can comprise: identifying one or more features in the received data; and comparing the one or more features to corresponding known features in sounds produced by the heartbeat of the user.

In any of the embodiments disclosed herein, the method can further comprise bandpass filtering the data.

In any of the embodiments disclosed herein, the method can further comprise zero-phase filtering the data.

In any of the embodiments disclosed herein, authenticating the user can employ a convolutional neural network (CNN).

In any of the embodiments disclosed herein, the data can be received at a smartphone.

Another embodiment of the present disclosure provides a system comprising at least one processor and a memory. The memory can comprise instructions that, when executed by the one or more processors, cause the one or more processors to perform one or more steps of any of the methods disclosed herein.

Another embodiment of the present disclosure provides a biometric authentication locking system, comprising a lock, a biometric identification system, and a transceiver. The biometric identification system can be any of the biometric identification systems disclosed herein. The biometric identification system can be configured to authenticate a user of the biometric identification system and transmit, with the transceiver, a signal to the lock instructing the lock to unlock.

These and other aspects of the present disclosure are described in the Detailed Description below and the accompanying drawings. Other aspects and features of embodiments will become apparent to those of ordinary skill in the art upon reviewing the following description of specific, exemplary embodiments in concert with the drawings. While features of the present disclosure may be discussed relative to certain embodiments and figures, all embodiments of the present disclosure can include one or more of the features discussed herein. Further, while one or more embodiments may be discussed as having certain advantageous features, one or more of such features may also be used with the various embodiments discussed herein. In similar fashion, while exemplary embodiments may be discussed below as device, system, or method embodiments, it is to be understood that such exemplary embodiments can be implemented in various devices, systems, and methods of the present disclosure.

### BRIEF DESCRIPTION OF THE DRAWINGS

The following detailed description of specific embodiments of the disclosure will be better understood when read in conjunction with the appended drawings. For the purpose of illustrating the disclosure, specific embodiments are shown in the drawings. It should be understood, however, that the disclosure is not limited to the precise arrangements and instrumentalities of the embodiments shown in the drawings.

FIG. 1A provides a block diagram of a biometric identification system, in accordance with some embodiments of the present disclosure.

FIG. 1B provides a schematic diagram of a continuous cardiac biometric (CCB) patch, in accordance with some embodiments of the present disclosure.

FIG. 1C provides a schematic illustration of a CCB patch on power, data hardware structure, and the real-time security system, in accordance with some embodiments of the present disclosure.

FIG. 1D provides a flow chart showing a biometric identification system from CCB patch to the control of mobile phones and door locks via a machine learning classification algorithm, in accordance with some embodiments of the present disclosure.

FIG. 2A provides an illustration of the heart valves and the phonocardiogram (PCG) principle in physiology.

FIG. 2B provides four representative raw PCG data of the time sequence on S1 and S2 peaks—S1: mitral and tricuspid valve closure, S2: closure of the semilunar (aortic and pulmonary) valves, in accordance with some embodiments of the present disclosure.

FIG. 2C provides a graph illustrating the pressure change in heart physiology, in which the order of occurrence is: (1) Isovolumetric contraction; (2) Ejection; (3) Isovolumetric relaxation; (4) Rapid intake; (5) Diastasis; and (6) Atrial systole, with important events: (i) A-V valve closes; (ii) Aortic valve opens; (iii) Aortic valve closes; and (iv) A-V valve opens.

FIG. 2D provides a graph of a normal ventricular volume in a cycle of occurrences in heart physiology.

FIG. 2E provides a plot of time series PCG data of systole and diastole synced with cardiac pressure and ventricular volume.

FIG. 3A provides a pre-processing overview of a flowchart for filtering stages of the raw CCB data, in accordance with some embodiments of the present disclosure.

FIG. 3B provides one second window plots of raw, band-passed, and zero-phase filtered data, in accordance with some embodiments of the present disclosure.

FIG. 3C provides a flowchart of an exemplary cardiac biometric classification.

FIG. 3D provides a confusion matrix of a trained model from a 20-participant data with an accuracy of 99.55%, in accordance with some embodiments of the present disclosure.

FIG. 3E provides an schematic of a CNN-based machine learning architecture for cardiac biometric classification, in accordance with some embodiments of the present disclosure.

FIG. 4A provides an illustration of a biometric identification system user unlocking a phone with the registered cardiac data as well as unlocking a door using the mobile application, in accordance with some embodiments of the present disclosure.

FIG. 4B provides a schematic flowchart of a control system and logistics of a door unlocking system, in accordance with some embodiments of the present disclosure.

FIG. 4C provides a schematic flowchart of a control device in hardware, in accordance with some embodiments of the present disclosure.

FIG. 4D provides screenshots of registered and unregistered users in a mobile application, in accordance with some embodiments of the present disclosure.

### DETAILED DESCRIPTION

To facilitate an understanding of the principles and features of the present disclosure, various illustrative embodiments are explained below. The components, steps, and materials described hereinafter as making up various elements of the embodiments disclosed herein are intended to be illustrative and not restrictive. Many suitable components, steps, and materials that would perform the same or similar functions as the components, steps, and materials described herein are intended to be embraced within the scope of the disclosure. Such other components, steps, and materials not described herein can include, but are not limited to, similar components or steps that are developed after development of the embodiments disclosed herein.

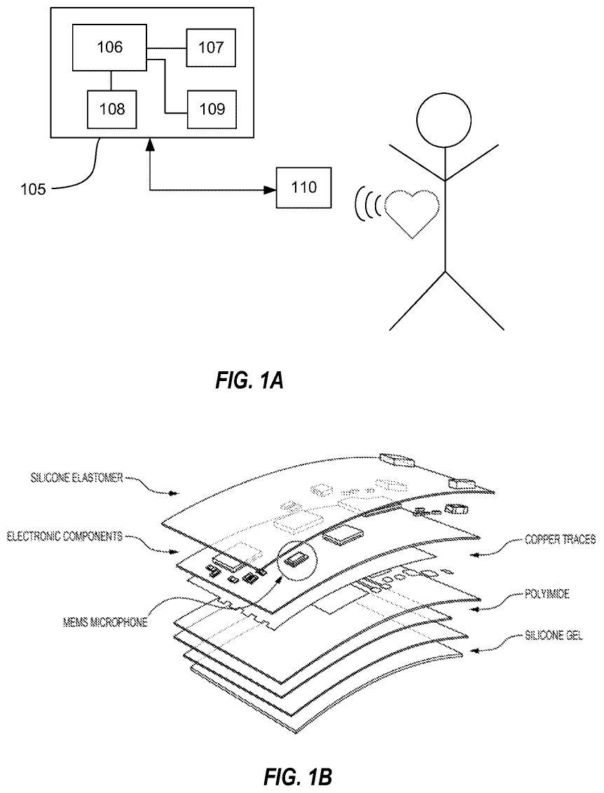

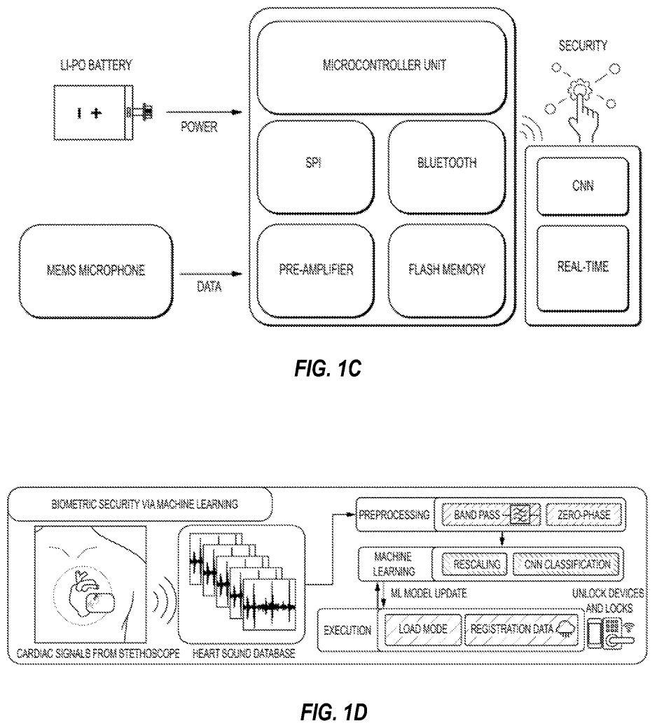

The present disclosure provides biometric identification systems and methods. In particular, embodiments of the present disclosure can utilize the unique sounds generated by the heartbeat of a user to identify and authenticate that user. As shown in FIG. 1, some embodiments of the present disclosure provide a biometric identification system comprising a sensor 110 and a controller 105. The sensor 110 can be configured to generate data corresponding to sounds generated by the heartbeat of the user. The controller 105 can be configured to receive the data from the sensor 110 and authenticate the user based, at least in part, on the received data.

The sensor 110 can be many different sensors known in the art capable of monitoring sounds generated by a heartbeat and generating corresponding sensor data. For example, in some embodiments, the sensor 110 can comprise one or more microelectromechanical system (MEMS) microphones. In some embodiments, the sensor 110 is a wearable sensor configured to be worn by a user. For example, the sensor 110 can be incorporated on a flexible patch, which can be referred to as a continuous cardiac biometric (CCB) patch, that can adhere to the user (e.g., proximate the user's chest). Mechanical aspects of the patch can be similar to and include one or more features of the patches disclosed in PCT Application No. PCT/US22/49141, entitled “Wearable Soft Electronics-Based Stethoscope,” which is incorporated herein in its entirety as if fully set forth below.

Some advantages of the CCB Patch's disclosed herein can come from their cross-disciplinary abilities and novel technologies. The devices' designs can employ precise nanomanufacturing to deliver critical attributes for its application, such as increasing its ambulatory aspects, ensuring device longevity, and optimizing component placement for proper auscultation. In addition, the meticulous nanomanufacturing of the CCB patches can open a new avenue for remote patient cardiopulmonary auscultation (FIG. 1A). FIG. 1B shows an exploded picture of one of the two mechanically soft, wireless components that comprise the system. The device can be a flexible patch, designed in KiCAD, with copper tracts across multiple layers connecting different board sections. The separate but linked layers of the circuit can be essential in manufacturing and packaging to reduce surface area, which can be helpful for usage on any patient who requires continuous auscultation with little to no pain. The ambulatory nature of the device can be a significant advantage, and it can come from the composing structure of the patch. Being a flexible device can provide the device with that malleability and bending. An additional part of its design can also allow for an increase in the device's ability to limit motion artifacts and bend through the elastomeric enclosure with an inner silicone-gel liner. The elastomer can help the skin conformality, ensuring compatibility with the delicate skin and highly curved anatomical features of users utilizing the device. The moisture vapor transmission rate (MVTR) of the other commercial medical tapes can be evaluated, and the resulting permeability of the medical tape used in the CCB patches can have the highest MVTR, clearly outperforming the other industry standard materials against which it was evaluated. The data from the long-term monitoring testing show consistent data points across two and a half hours of recording. This means that the patch can be permeable enough not to irritate over a long period of wearing the device. The before and after image where the device was placed on the skin shows that little to no disturbance had occurred. The exemplary passage of all these tests indicates that the CCB patches can endure most of the environments they could experience while on a patient. In some embodiments, the ambulatory nature of the devices can allow for increased quality data because of its ability to move with the user instead of the user moving the microphone. In some embodiments, the flexibility and skin-conformal contact by elastomer layers can be assist in continuous biometric identification. The ambulatory nature, in combination with continuous recording, can create the ability to seamlessly record and identify individuals across several layers of identification and throughout an extended period. This can eliminate the need to re-scan at every access point where another biometric would require yet another scan. Continuous recording can identify unauthorized individuals with a variation in cardiac cycles, which do not change over extended periods, making it more difficult to imitate than one-time biometric scans. To replicate the sound, one would likely need to have an exact replica of the heart and chest cavity because the sound is made from the valves in the heart opening and closing and reverberating through the body.

The controller 105 can be many different controllers known in the art, including, but not limited to, microcontrollers, central processing units, smartphones, laptop computers, tablets, and the like. In some embodiments, the controller 105 can comprise one or more processors 106 and one or more memories 107. The one or more memories 107 can individually or collectively comprise logical instructions that, when executed by the one or more processors 106 (either individually or collectively) cause the controller 105 to carry out the various functions disclosed herein. For example, the controller 105 can receive data from the sensor(s) 110 and authenticate the user by identifying one or more identifying one or more features in the data received from the one or more sensors 110 and comparing the one or more features to corresponding known features in sounds produced by the heartbeat of the user. Thus, the controller 105 can make use of the known unique sounds generated by the heartbeat of a user and determine if the sounds received from the sensor 110 match the known heartbeat data.

The controller 105 can be configured to perform certain preprocessing of data received from the sensor 110 to perform the authentication process. This can include bandpass filtering and/or zero-phase filtering the received data. Bandpass filtering can occur over various frequency ranges in accordance with various embodiments of the present disclosure. In some embodiments, bandpass filtering can occur over a frequency range of 20 Hz to 250 Hz, though the disclosure is not so limited and can include other ranges. Zero-phase and bandpass filtering can be conducted to uniquely distinguish each heart signal into unique waveforms in second windows for a machine learning algorithm, all integrated with the mobile application.

The controller 105 can comprise various hardware and/or software components to perform its various functions, including, but not limited to, an analog-to-digital converter, a serial peripheral interface (SPI), a pre-amplifier, flash memory, a transceiver (wired or wireless, e.g., Bluetooth) 109, a battery/power supply, a data acquisition unit 108, and the like.

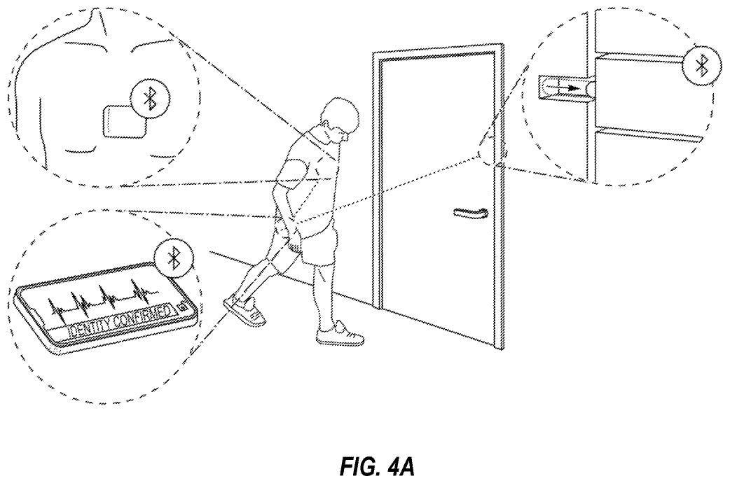

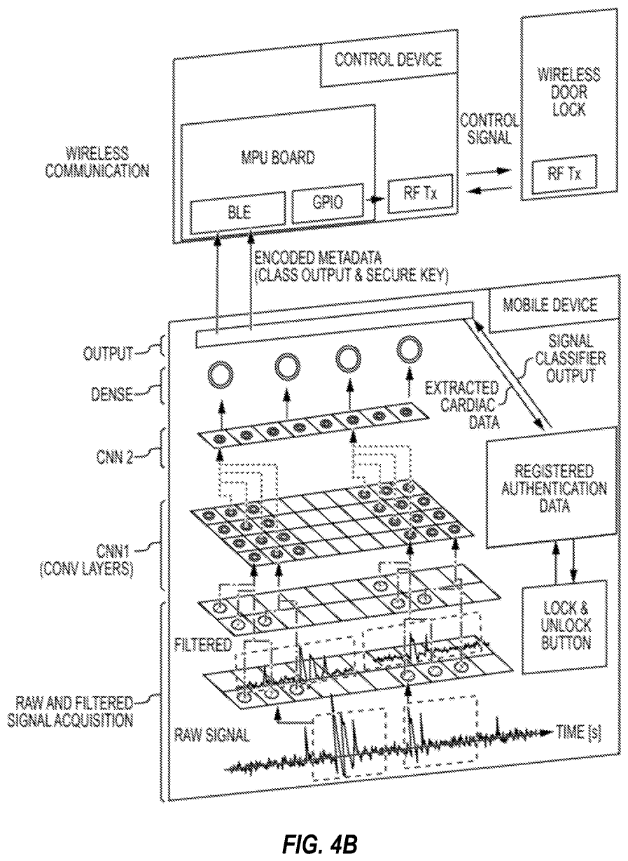

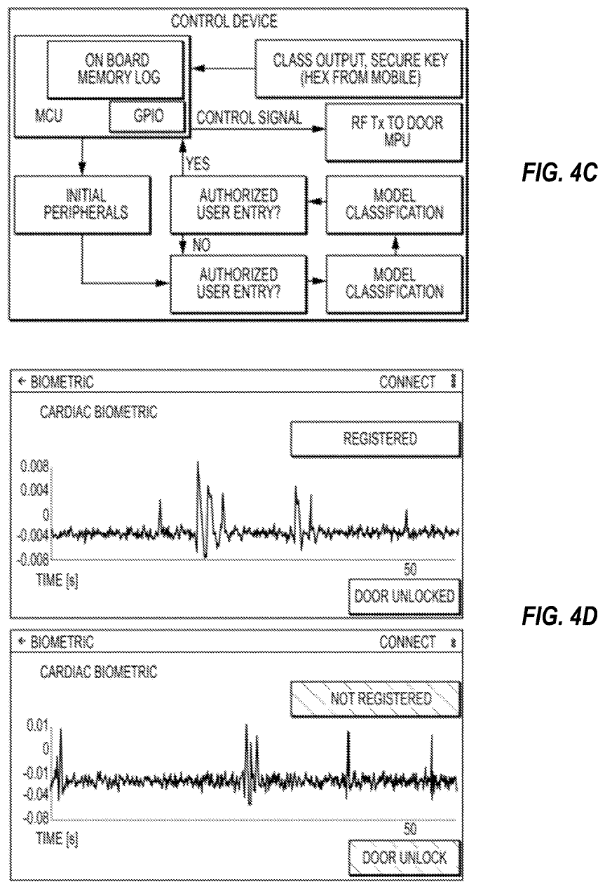

In some embodiments, the controller (or a portion of the controller) 105 can be mechanically coupled to the sensor 110. For example, the controller (or a portion thereof) 105 can be integrated into the flexible patch. In some embodiments, a portion of the controller 105 can be integrated into the flexible patch and another portion of the controller 105 can be located remotely, e.g., a smartphone, and be configured to communicate with the patch, e.g., wirelessly. Such an embodiment can allow for various applications based on the authentication of the user. For example, as shown in FIG. 4A, the biometric authentication system can be used to control a lock. If the controller 105 is able to authenticate the user, the controller 105 can send a signal (e.g., wireless signal) to the lock instructing the lock to transition from a locked position to an unlocked position.

Applying the circuitry to the board on the patches that can form part of the controller 105 can be quick, efficient, and, effective. The CCB Patches can be powered by a small, 40 mAh lithium-ion polymer battery, and for a guided battery connection, the circuit's power pads can be connected to the battery. Battery location can ensure gentle placement on the curved skin of the chest by being secured atop the device. The battery can enable it to auscultate cardiac activity by a Bluetooth Low Energy (BLE) system-on-a-chip (SoC) and associated set of sensors for the auscultation of cardiac activity. The analog signal acquired from the sensor 110 (e.g., MEMS microphone) can travel through the pre-amplifier. Then it can move to the ADC to get converted to a digital signal to feed the processor (e.g., BLE microcontroller) to send data wirelessly via Bluetooth to mobile devices (FIG. 1C). The raw signal acquired from the device can then be processed through the convolutional neural network (CNN) (discussed below) training with cardiac activities logging to physicians and finally fed into the secondary authentication using cardiac sounds. The extracted features can be trained in the model to authenticate the individual. This process flow is simplified in FIG. 1D. The device's standard biometric qualities, ambulatory nature, remote and continuous recording, very low possibility of a change in heart sound for an individual, and the near impossibility of forgery can combine to create a secure, easy-to-use biometric security system.

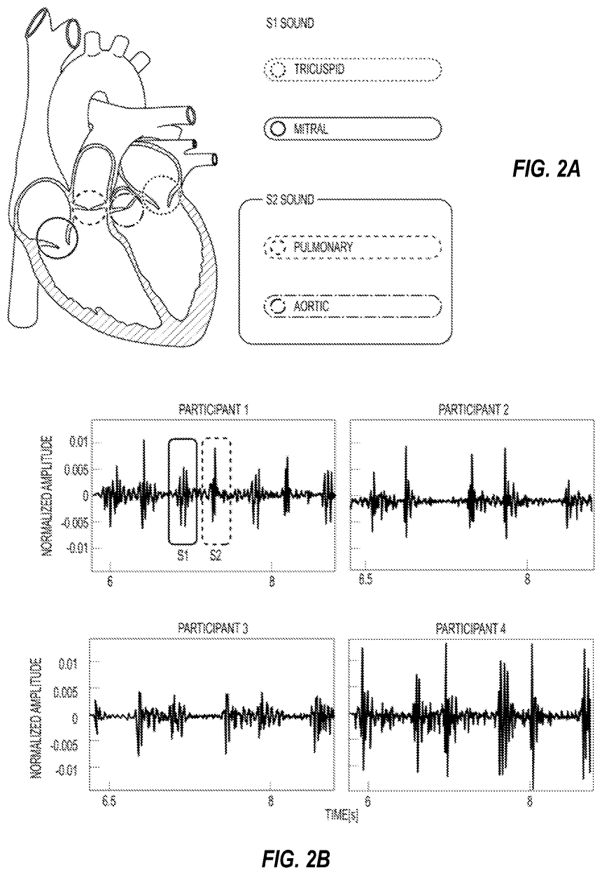

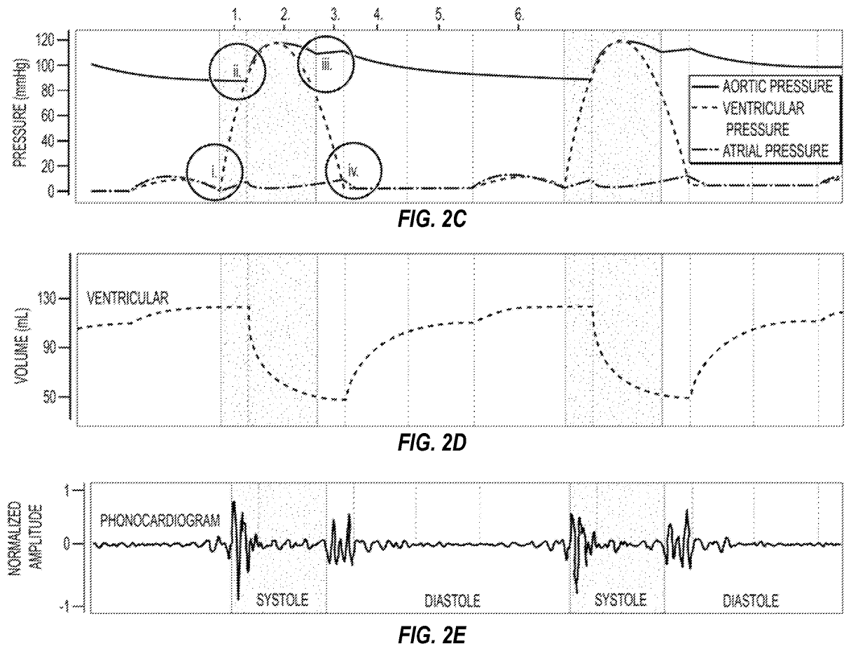

As discussed above, the systems disclosed herein can analyze cardiac sounds and the characteristics that make them unique to create the basis of this biometric. The opening and closing of the heart's valves produce cardiac sounds. The heart has several distinct sounds, including S1, S2, S3, and S4. In some embodiments, the points of interest for the device can be S1 and S2 for normal subjects. The S1 sound is produced by the mitral and tricuspid valves closing, as shown in FIG. 2A. This period of the cycle is known as systole. When the pulmonic and aortic valves shut during diastole, the S2 sound is produced, which is the louder of the two, and this is the beginning of diastole. The average duration of systole and diastole are 0.35 seconds and 0.45 seconds, respectively, totaling a cardiac cycle lasting approximately 0.8 seconds shown in 4 representative participants' data from FIG. 2B. The complete cardiac cycle as S1 and S2 coordinate with pressure and volume changes can be seen in FIGS. 2C-D. The cardiac sounds have a frequency range of from 20 Hz to 220 Hz.

The device's audio resolution can be subtle, such that specifics concerning the noises produced by the heart may be identified. Those specifics can include several aspects of the cardiac sound, like magnitude, shape, size relativity between S1 and S2, and standard deviations in the timing duration of a cardiac cycle. Other factors influencing cardiac sound can be how the sound reverberates within the heart and chest cavity. Significant individual variation is caused by the heart's activation order, conductivity, and heart mass orientation. For example, an individual with a fatty heart could have quieter heart sounds, while an individual with a large chest cavity could have a deeper frequency sound. The unique characteristics of a user's heartbeat can be used in some embodiments of the present disclosure to biometrically identify/authenticate the user.

Once the variability of an individual's heart rate is measured and quantified, the next step begins. The raw data set from the CCB Patch can be first run through digital signal processing. Two filters can be applied to it, including a zero-phase filter with a 3rd order Kaiser window for magnitude normalization and a bandpass filter set to cut off frequencies outside of the frequency range of the heart, which can be 20-250 Hz. After the filters are applied, and a new, clean data file can be produced, the information can be sent to the machine learning program (discussed below). After the precise and high-quality data is gathered using the CCB patch and cleaned through extensive digital signal processing, the computer can generate an individual profile with the aforementioned specifications wired in it. If the algorithm notices the known characteristics in an incoming data flow, it can identify the owner of that specific data set. The heart sound in time series, with S1 and S2 visible and distinguishable, is presented in FIG. 2E synced with varying pressures and ventricular volumes for two complete cardiac cycles. This time-series plot is what machine learning takes after filtering away outside noise interference. The data set's purity reflects the efficacy of the device's digital signal processing and noise suppression algorithms.

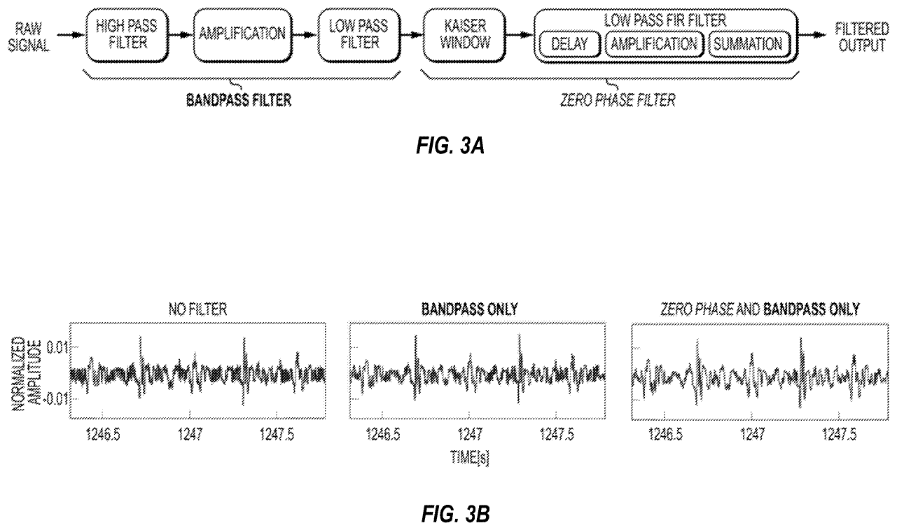

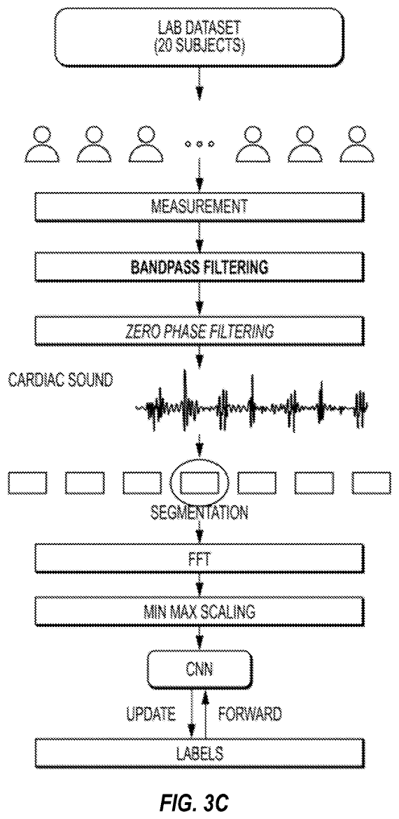

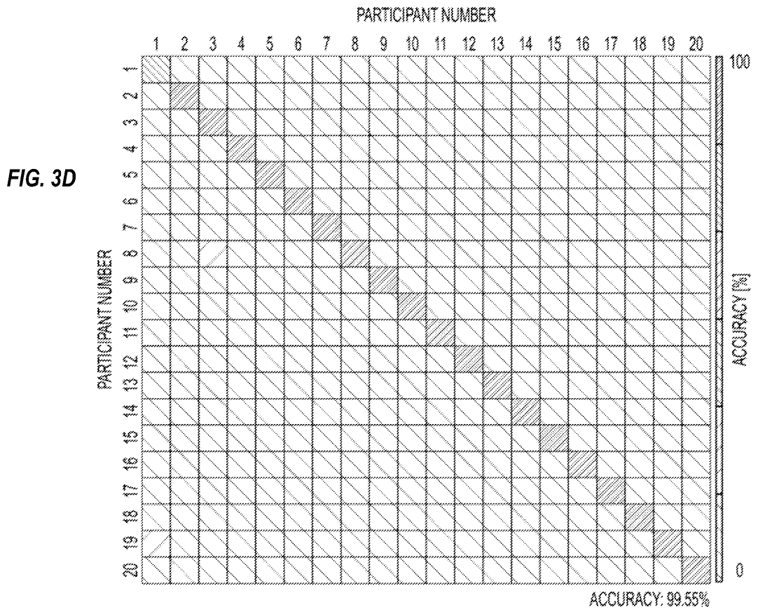

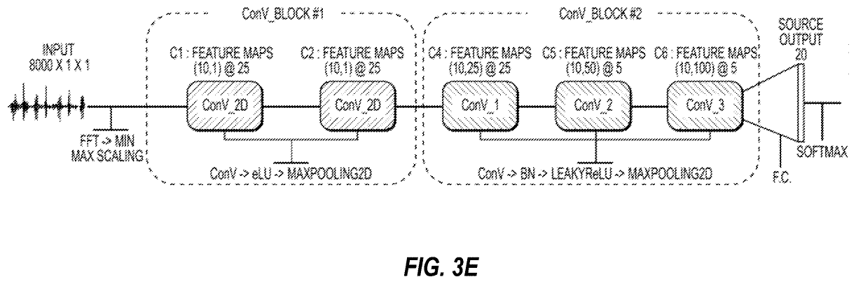

To perform the authentication process, in some embodiments, the controller can utilize various machine learning (ML) algorithms, as discussed below. For pre-processing cardiac data from the CCB patch, bandpasses of 20 Hz to 250 Hz and zero-phase filtering (FIG. 3A) were done to reduce noise in the signal and preserve time and phase shift issues which can be useful in time-series-based ML training. Details of the zero-phase filtering are shown in FIG. 3A, and FIG. 3B shows the one second window of an example cardiac signal from the CCB patch with raw, band-passed, and zero-phase filtered graphs. While bandpass can eliminate the DC offset caused by the device, it may not entirely reduce the baseline noise, which zero-phase filters out with a phase shift of zero for all frequencies. In an exemplary convolutional neural network (CNN) architecture, data from 20 subjects in a lab dataset were utilized. The dataset was divided into three segments: 60% for training (40,075 epochs), 20% for validation (13,625 epochs), and 20% for the test (13,625 epochs). During each training iteration, the CNN network parameters' weights were adjusted based on the model's training validation accuracy. Most of the hyperparameter values, such as learning rate, kernel size, filter count for each convolutional layer, and units for each dropout, were determined using a random search approach.

Ultimately, the model with the highest validation accuracy was selected as the optimal model, though the present disclosure should not be interested as limited to this optimal model. The performance of this top model was assessed based on the prediction accuracy of the test dataset. Consequently, the exemplary CNN architecture employed (3,1) pool size of 2D-max pooling, 25 filters, and (10,1) kernel size for the two 2D-convolutional layers in the initial block. Batch normalization was incorporated to mitigate overfitting. For the second block, 25 filters and sequential kernel sizes of (10,25), (10,50), and (10,100) were used for the three individual convolutional cells (Conv_1,2,3), along with a (3,1) pool size of 2D-max pooling (refer to FIG. 3C-D). A series of layers in the machine learning model was applied, as illustrated in FIG. 3E. The model was trained to ensure that each participant's heart sound waves had different patterns and forms for each S1 and S2 pair segment in the time series. Because the participants' average beats per minute were about 60, 120 samples with the input of 2 seconds were sent to each participant's class. The confusion matrix of each participant's different waveform trained in the model is shown in FIG. 3D, demonstrating that the machine correctly detects each participant's heart sounds.

As demonstrated by the proposed biometric accuracy or correct recognition rate of 99.55%, the heart sounds biometric offers a solution to all these difficulties, albeit having an error rate of 0.45 percent. As compared with other convention devices, the systems disclosed herein show the best performance in accuracy. The suggested heart biometric outperformed the estimated error rate of fingerprint, signature, and voice recognition. There is an extremely little chance that a person's heart sound will alter throughout a recorded time. Heart sounds are difficult to fake unless an unauthorized user attempts to clone a heart identical to another person's and set it within a chest cavity that reverberates similarly. Compared to the issues that facial recognition software, fingerprint identification, and other biometric security measures have brought up, measuring an individual's heart sound may be a far less invasive alternative. The capacity to continuously acquire an individual's heart sound is the most significant and evident benefit because it creates identification across many layers of security. The goal is to eliminate the requirement to re-scan a biometric at every entry point necessitating a new scan. The CCB Patch is an efficient, adaptive, unobtrusive, and accurate technology that analyzes an important biometric in the body—heart sounds.

Additionally, in some embodiments, the system can be included as part of a biometric locking system. FIG. 4A shows the overall system of an exemplary security system using a CCB. Data transmission structure can be as follows: wearable devices on the user's sternum, mobile devices, and wireless door lock systems. In this example, three devices can work sequentially for secure biometric authentication: a lock with an RF receiver mounted with a linear actuator to unlock the lock, an RF remote integrated with the BLE development kit. Finally, a mobile device can be the primary device to compute the authentication system. The system can apply security functions through Android Keystore API in Jetpack android studio library for the encryption and decryption (or store and access) of biometric data input/output stream, allowing for the secure processing of the data. CNN model can be embedded in the Android application with TensorFlow. The CNN model can extract the S1 and S2 data in real time for the user's feature detection and authentication. After applying the CNN model, the new user's processed data can be registered for authentication.

For the performance and secure file stream structure of the CCB system, a signal classifier mechanism was developed to evaluate CNN's continuous input cardiac data. The classifier can be based on sequential matching and anomaly detection that can comprehensively predict both class labels and the similarity of signal features. App backend can decrypt the model and compares the user signal features. The signal classifier output can be wirelessly communicated as metadata to the control device, as shown in FIG. 4B. Encoded metadata of continuous packets from mobile device advertise through read/writable BLE structure on individual UUID characteristics. Since the output from the mobile device is a decision-making signal, it can be encoded securely. A key pair decoder can only decode it in the control device firmware. Inside the control device, authentication of model output can be used as decision-making signal data, as illustrated in FIG. 4C. The firmware structure in the microcontroller can decode the signals and validate the model classification. With an embedded validation algorithm, the control device can record the success log to the internal board and send a control signal to the wireless door lock. Also, the control device can send a writable byte signal to the mobile device for an additional record logging of successful authentication shown in FIG. 4D. Lastly, the wireless door lock can receive the paired control device's signal and control the motor driver to lock or unlock the door. The CCB interface can show a secure hardware and software interface applicable to wide-ranging applications. In addition, because the second user was not one of the 20 training data on the ML algorithm, the CCB system identified as not a registered individual to attempt door unlock. If we expand the sample size, the accuracy can rise and keep around 100% accuracy. Embodiments of the present disclosure can find additional applications using wearable cardiac-biometric systems, including, but not limited to, confidential file transfer, bank security, 2-factor authentication, and secured remote patient monitoring.

### Experimental Section

Below certain experimental results are described regarding some exemplary embodiments. The embodiments disclosed herein, however, are exemplary only and should not be construed as limiting the scope of the present disclosure.

Disclosed herein is the development of wearable soft CCB patches for recording heart sounds and demonstrating biometric locking mechanisms. The heart sounds of twenty individuals were collected to a clean dataset. The signal processing includes two signal processing stages to eliminate any interference in the data. A CNN-based machine learning algorithm, developed in this work using the data, shows an accuracy of 99.55%. This result demonstrates the potential of the CCB patch to outperform current biometric systems in terms of practical usability and security. It is shown that the wearable patch-enabled biometric system can be used for digital security by replacing passwords and enhancing safety in virtual reality transactions. The use of biometrics in this manner offers the key advantages of being difficult to replicate and not requiring good user memory or a physical key. Overall, the CCB patch demonstrates its fast, hands-free, and remote identification capabilities

Device Fabrication: A flexible printed circuit board (FPCB) was designed using kiCAD software for the wearable stethoscope sensor. The entire device was 53 mm×25 mm at its widest points. The copper tracts of the FPCB ended on the board's surface at a copper pad, and the nanocircuitry was placed on its respective set of copper pads and melded on using solder paste and a hot plate. This meticulous process was completed to ensure the longevity of the device's functioning. A layer of a soft elastomer gel was used as a base adhesion layer for the wearable stethoscope to the skin. A 20-gram mixture of the Eco-flex gel A & B was poured onto the 150 mm diameter petri dish and spin-coated at 1000 rpm for 5 seconds. This ensured that the even layer touches the skin without deformation to the circuit. The rough boundary was cut out from the cured gel layer in the petri dish, and the integrated circuit board was placed on top of the gel layer. A hole was sliced in the middle of the microphone island to allow sound to permeate through the elastomer layer. Another layer of a soft silicone elastomer was created by mixing a 1:1 ratio of Eco-flex 30 A & B and pouring it on top of the entire circuit board, covering the gel layer's edges to encapsulate the device entirely. Lastly, high-tack silicone gel was used on a fabric layer, spin-coated at 1000 RPM for 30 seconds cured on a 60° C. hot plate for 15 minutes. The fabric was cut out in a circle on top of the encapsulated microphone island for better pressure applied to the microphone.

Mechanical Study: The following mechanical tests were performed on the device to ensure its performance across all types of environments: bend/stretch testing, SNR testing, contact pressure testing, waterproof testing, permeability testing, and irritability/duration testing. The stretch/bend testing was conducted using the ESM303, Mark-10 machine; it stretched the device while resistivity was recorded to see if there was any effect on the device's efficacy. The same was done for the bend test. Additionally, the device's attachment material was tested for its ability to maintain a high enough level of pressure in skin contact and deliver an acceptable signal-to-noise ratio (SNR). The waterproof testing was the simplest of all the tests. It entailed starting the recording, submerging the device, taking it back out, and seeing if it could continue to record afterward. Finally, the permeability testing was conducted by filling uniform containers to the brim with water and sealing them using different types of medical coverage, with an open container as a control. Water evaporates faster out of the containers filled by a medical range with higher permeability, so after seven days untouched, the material with the most increased permeability would be the emptiest. Finally, the irritability/duration testing was conducted by collecting cardiac data across 2.5 hours and examining the skin where the device was placed (to evaluate irritability) and the quality of the data collected.

Data Collection & Filtering: The CCB Patch was mounted slightly to the left of the sternum for optimal cardiac sound collection and was tested on 20 different participants. The duration for each session was approximately 60 seconds. The subject was stationary and seated for the collection process. The patch acquired raw cardiac signals and sends buffered data wirelessly to a computer or mobile device. The signals were synched with timestamps and saved through files such as CSV, and ready to be analyzed. First, the raw signal underwent signal processing to produce a pure form of the training and CNN classifier data. After signal processing and smoothing for feature extraction, the preliminary digital sound data iwass pre-processed for machine learning. Lastly, the signal can be analyzed as an individual profile in the CNN model.

Classification of Heart Sounds: Digitized sound data is recorded at 4,000 samples per second, and a professional human analyzer segments the corresponding data for training. The pieces of data were then fed into a machine learning algorithm (TensorFlow by Python), and profiles are created for everyone. In their profile, the machine is trained with information about their heart sound, how it can vary across time, and what is specific about it to them. To maintain a balanced dataset during the training process, the number of samples for each class was adjusted to be approximately equal, thus minimizing classification bias. The model's architecture was devised through a series of iterative refinements, informed by prior research. The model was designed based on the input data of a CNN architecture, specifically for time-filtered signal data. The CNN architecture inputs consisted of 2-second raw signal data, having a size of 8000×1. To pre-process the dataset of CNN architecture, Fast Fourier Transform (FFT) and Min Max Scaling were used to extracting features. The chosen non-linear activation function was the Leaky Rectified Linear Unit (Leaky ReLU). The CNN architecture was optimized using the ADAM optimizer (learning rate=0.001, ß1=0.9, ß2=0.999), with a batch size of 64. To avoid overfitting, early stopping was implemented by randomly excluding 30% of the data from the training set and designating it as the validation set at the onset of the optimization phase. If the validation loss ceased to improve, the learning rate was decayed by a factor of 5. The training was terminated if there were two consecutive instances of learning rate decay without any observed improvement in the network performance on the validation set. The CNN architecture incorporated two distinct convolutional blocks. The initial block consisted of a pair of 2D convolutional layers, batch normalization, and a 2D max pooling layer. The subsequent block featured three separate convolutional cells (Conv_1,2,3). Each individual convolutional cell (Conv_N) contained a convolutional layer, a batch normalization layer, a Leaky ReLU layer, and a max pooling layer. The Convolutional Neural Network's final output was a 20×1 vector, which was subsequently passed through a softmax layer, resulting in the predicted class corresponding to one of the twenty participants.

Biometric Authentication: A real-time biometric authentication platform was developed to process real-time CNN models in a mobile device. A secure and fast deep learning framework with Tensorflow (v2.8) and Tensorflow lite processes the output of the CNN model. TF was used for deploying our designed dataflow structure to compute the output array by processing acquired signal data from CCB. TF lite serialized toolkit is embedded in the Android Kotlin Application (Kotlin v 2022.1.7) and facilitated the implementation of the CNN algorithm. The signal processing system used in ML training was converted to C++ for the fast framework of real-time signal processing to handle real-time input. The custom classifier mechanism comprehensively analyzes segments sequentially and detects S1, S2, feature detections, and anomalies of the input filtered signal along with CNN classes. With user input of the lock & unlock button, the extracted cardiac data is evaluated with a comparison of registered authentication data and generates a signal classifier output. The output layer of the CNN model is developed for user biometric data in real-time. The feature extracted cardiac data assessed through trained and registered authentication data inside the app. The platform utilized security procedures with Android Keystore API in Jetpack android studio library (Compose v1.1) for model encoding/decoding, critical pair matching, and modeling loading process. The primary (master) key was generated for individual app releases and recorded for additional protection. The output sends wirelessly as metadata with byte algorithms that can only be decoded to the particular control device. The metadata received on the control device board as a hex-type with nRF52832 and BLE platform. The control device receives only correct peripherals signals (matching specific service UUID and byte decoder) from a mobile device and decodes the output with the received secure key. The embedded assessment algorithm in nRF52832 evaluates the class model and makes authentication decisions. Lastly, the control device transmits the control signal to the door RF receiver integrated with the linear actuator (Actuonix). For security purposes, the control device records the log of the successful trials on board and returns the success signal to a mobile device by writable characteristic UUID. This allows future investigation of tracking authentication history. The paired control device can only initiate the final wireless door lock system. The door locker receives specific critical information to unlock the door.

It is to be understood that the embodiments and claims disclosed herein are not limited in their application to the details of construction and arrangement of the components set forth in the description and illustrated in the drawings. Rather, the description and the drawings provide examples of the embodiments envisioned. The embodiments and claims disclosed herein are further capable of other embodiments and of being practiced and carried out in various ways. Also, it is to be understood that the phraseology and terminology employed herein are for the purposes of description and should not be regarded as limiting the claims.

Accordingly, those skilled in the art will appreciate that the conception upon which the application and claims are based may be readily utilized as a basis for the design of other structures, methods, and systems for carrying out the several purposes of the embodiments and claims presented in this application. It is important, therefore, that the claims be regarded as including such equivalent constructions.

Furthermore, the purpose of the foregoing Abstract is to enable the United States Patent and Trademark Office and the public generally, and especially including the practitioners in the art who are not familiar with patent and legal terms or phraseology, to determine quickly from a cursory inspection the nature and essence of the technical disclosure of the application. The Abstract is neither intended to define the claims of the application, nor is it intended to be limiting to the scope of the claims in any way.

1. A biometric identification system comprising:
a wearable skin-conformal sensing patch comprising one or more sensors configured to generate data corresponding to sounds generated by the heartbeat of a user; and
a controller configured to receive the data and authenticate the user based, at least in part, on the data by:
identifying one or more user unique features in the received data; and
comparing one or more of the user unique features in the received data to a dataset of known features in sounds produced by the heartbeat of the user.

2. The biometric identification system of claim 1, wherein one or more of the sensors comprise a microelectromechanical system (MEMS) microphone.

3.-4. (canceled)

5. The biometric identification system of claim 1, wherein the user unique and known features correspond to heart sounds of the user selected from a group consisting of S1, S2, S3, S4, and a combination thereof.

6. The biometric identification system of claim 1, wherein the user unique and known features correspond to one or more of magnitude, shape, size relativity between S1 and S2 sounds, and standard deviations in a timing duration of a cardiac cycle of the user.

7. The biometric identification system of claim 1, wherein:
the wearable skin-conformal sensing patch is a layered wearable skin-conformal sensing patch comprising:
a flexible printed circuit board layer having the one or more sensors configured to generate data corresponding to sounds generated by the heartbeat of a user;
a gel layer configured for direct contact with skin of the user, with a thickness and flexibility to maintain the direct contact without subjecting the flexible printed circuit board layer to an extent of deformation that affects the performance of the flexible printed circuit board layer; and
an elastomeric enclosure layer encapsulating the flexible printed circuit board layer and the gel layer, the elastomeric enclosure layer configured for skin conformality and compatibility with surface anatomical features of the skin in contact with the gel layer;
wherein the elastomeric enclosure layer and gel layer are configured to limit motion artifacts and bend of the patch; and
the controller is further configured to bandpass filter the data.

8. A biometric identification system, comprising:
a layered wearable skin-conformal sensing patch comprising:
a flexible printed circuit board layer having one or more sensors configured to generate data corresponding to sounds generated by the heartbeat of a user;
a gel layer configured for direct contact with skin of the user, with a thickness and flexibility to maintain the direct contact without subjecting the flexible printed circuit board layer to an extent of deformation that affects the performance of the flexible printed circuit board layer; and
an elastomeric enclosure layer encapsulating the flexible printed circuit board layer and the gel layer, the elastomeric enclosure layer configured for skin conformality and compatibility with surface anatomical features of the skin in contact with the gel layer;
wherein the elastomeric enclosure layer and gel layer are configured to limit motion artifacts and bend of the patch; and
a controller configured to:
receive the data;
zero-phase filter the received data; and
bandpass filter the zero-phase filtered data over a frequency range of from 20 Hz to 250 Hz to cut off frequencies outside of the frequency range of the heart.

9. (canceled)

10. The biometric identification system of claim 8 further comprising:
an analog to digital signal converter configured to receive analog data from one or more of the sensors and convert the analog data to digital data to be processed by the controller.

11. The biometric identification system of claim 8, wherein the controller is further configured to:
provide the filtered data to a convolutional neural network (CNN) to authenticate the user by:
identifying one or more user unique features in the provided data; and
comparing one or more of the user unique features in the provided data to a dataset of known features in sounds produced by the heartbeat of the user

12. The biometric identification system of claim 11, wherein the CNN is trained with data collected from the user.

13. (canceled)

14. The biometric identification system of claim 11, wherein the controller is a component of a smartphone configured to receive data from one or more of the sensors wirelessly.

15. (canceled)

16. A method of authenticating a user by evaluating the heartbeat of the user comprising:
receiving data indicative of sounds generated by the heartbeat of the user using the biometric identification system of claim 8; and
authenticating the user based, at least in part, on the data.

17.-28. (canceled)

29. A system comprising:
a biometric identification system comprising:
a layered wearable skin-conformal sensing patch comprising:
a flexible printed circuit board layer having one or more sensors configured to generate data corresponding to sounds generated by the heartbeat of a user;
a gel layer configured for direct contact with skin of the user, with a thickness and flexibility to maintain the direct contact without subjecting the flexible printed circuit board layer to an extent of deformation that affects the performance of the flexible printed circuit board layer; and
an elastomeric enclosure layer encapsulating the flexible printed circuit board layer and the gel layer, the elastomeric enclosure layer configured for skin conformality and compatibility with surface anatomical features of the skin in contact with the gel layer;
wherein the elastomeric enclosure layer and gel layer are configured to limit motion artifacts and bend of the patch; and
a controller configured to:
receive the data;
zero-phase filter the received data; and
bandpass filter the zero-phase filtered data over a frequency range of from 20 Hz to 250 Hz to cut off frequencies outside of the frequency range of the heart;
a processing platform comprising at least one processor; and
a memory comprising instructions that, when executed by the processing platform, cause the processing platform to perform the method of claim 16.

30. A biometric authentication locking system comprising:
a lock;
the biometric identification system of claim 8; and
a transceiver,
wherein the biometric identification system is configured to authenticate a user of the biometric identification system and transmit, with the transceiver, a signal to the lock instructing the lock to unlock.
